(19) United States
(12) Patent Application Publication
(10) Pub. No.: US 2009/0216172 A1
(43) Pub. Date: Aug. 27, 2009

(54) Pre-moistened cotton swabs

(76) Inventor: Mina Houtan, Egg Harbor Township, NJ (US)

Correspondence Address: Mina Houtan, Ph.D., 300 Central Avenue, Egg Harbor Township, NJ, 08234, US

(21) Appl. No.: 12/070,732

(22) Filed: Feb. 21, 2008

(51) Int. Cl. A61F 13/15; B29C 65/72; B01J 19/00

(52) U.S. Cl. 604/1; 156/281; 422/40

(57) ABSTRACT

A process for manufacture of an improved cotton swab product for use in personal hygiene, application or removal of cosmetics, or application of medication. Cotton swabs today are sold in packages containing hundreds of individual swabs that can come into contact with germs once the package is opened. This improved invention will prevent contamination of the swabs because they will be individually wrapped. Further, this improved invention will contain a saline solution sprayed into each cotton tip of the swab during the processing stage which will allow the user to clean their ears or other body parts much easier than using just a dry cotton swab. Dry swabs do not properly clean the ears and with this improved invention of a pre-moistened swab, personal hygiene will be much easier and more thorough.

### BACKGROUND OF THE INVENTION

1. Field of the Invention

This invention relates to soft, absorbent, pre-moistened cotton swabs that are used for personal hygiene tasks, particularly for cleaning the ears.

2. Background Art

The design of the cotton swab has had significant advances since its invention in the 1920's. In the early design stages wooden sticks were used, but eventually the spindles were made of paper and now some are even made of plastic because it improves the flexibility of the spindle during manipulation.

The most popular use for cotton swabs is infant care, however cotton swabs are commonly used in many other areas. For example, doctors today use cotton swabs during throat cultures while laboratories use them for microbiological cultures. Other uses may be for cleaning small parts on machinery or equipment, make-up application, painting or medication application. Most of the cotton swabs used in industry are commonly made with wooden spindles for greater rigidity and have a cotton tip on only one end of the spindle.

Ear wax is a sticky substance that can be difficult to remove with a dry cotton tipped swab. Often times when cotton swabs are used, particularly in infants, they have to be moistened with water prior to their use, therefore this improved version of the cotton swab is of one that is pre-moistened with a solution that will allow the user to clean their ears with ease.

### BRIEF SUMMARY OF THE INVENTION

This improved invention of the cotton swab will offer just enough moisture to properly clean ears and other delicate parts of the body where cotton swabs can be used. This improved cotton swab will be pre-moistened with a normal saline solution and packaged individually to preserve the moisture of the product. This cotton swab will be made with a plastic spindle to remain impervious to the saline solution and it will have a cotton tip on both ends of the spindle.

### BRIEF DESCRIPTION OF THE DRAWINGS

FIG. 1 The plastic spindle measuring 75 mm or 3 inches in length

FIG. 2 Wad of absorbent cotton which is adhered to the tip of both ends of the spindle with a small amount of adhesive

FIG. 3 Plastic covering into which the finished swab will be packaged. This plastic covering will be heat sealed at both ends and will preserve the moist swab inside. This is a thin plastic film similar to the wrap on lollipops.

FIG. 4 The finished product as it will be packaged and sold

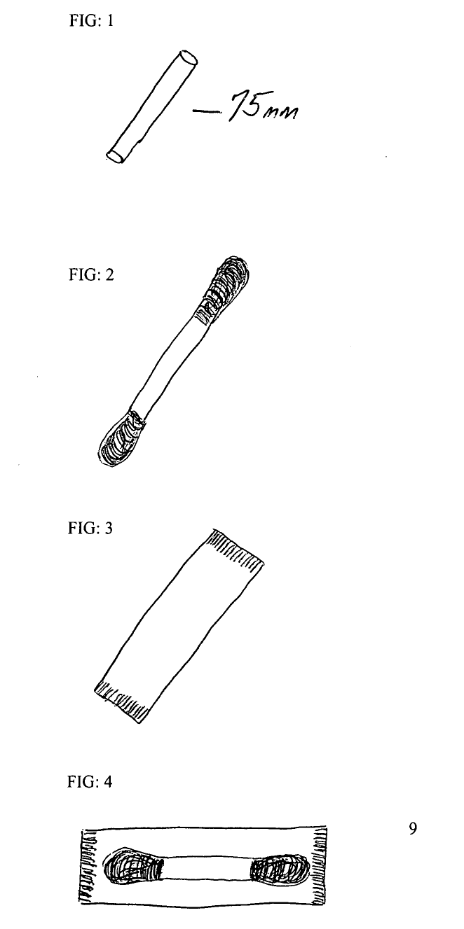

### DETAILED DESCRIPTION OF THE INVENTION

The advantages and features of this present invention will be more fully appreciated through the following detailed description. The first step in this invention is to create the plastic spindle which is done by an extrusion molding process. The plastic resin and other additives are blended, heated, and extruded through a die similar to the way spaghetti strands are made. These plastic spindles will measure about 3 inches in length. Once they are complete they would be loaded into hoppers in preparation for the application of the cotton tips.

The hopper onto which the spindles are loaded will drop each spindle into a conveyer that will travel to the next process. The spindles will be held in place individually and rotate as they reach the adhesive process. Adhesive will be applied to both ends of the spindle and continue to the cotton application stage. Approximately 0.05-0.1 g of cotton will be spun on both ends and continue in a process that will compress and smooth the cotton on the tips and shape them, completing the dry process.

Chemical coating is applied to prevent mildew and then the dry swab is sprayed with saline solution before heading to the wrapping area. The swabs will continue through the wrapping process and each individual swab will be wrapped in a plastic outer wrapping similar to lollipop wrapping to prevent the swab from drying out.

The finished product will proceed to the packaging station where they will be sent through a counting wheel and the wheel will count out the exact number of individual swabs for final packaging.

1. An improved process for manufacturing a cotton swab product comprising of a plastic spindle 3 inches long with adhesive on both ends to which cotton is attached and shaped to 0.05 to 0.1 g of cotton in the shape similar to the shape of an eye. This cotton swab will be treated chemically to prevent molding and a small amount of saline solution will be applied during the swab making process to complete the final product.

2. The cotton swab product of claim number one will be individually wrapped for preservation of moistened swab.

3. The cotton swab of claim number one will contain a chemical product for prevention of mold and a small amount of saline solution on each cotton tip.
